(19) United States
(12) Patent Application Publication
(10) Pub. No.: US 2010/0152712 A1
(43) Pub. Date: Jun. 17, 2010

(54) FLUID PROCESSING SYSTEM AND COLLECTING DEVICE THEREOF

(75) Inventor: Jyh-Wen Wu, Hsinchu City (TW)

Correspondence Address: QUINTERO LAW OFFICE, PC, 615 Hampton Dr, Suite A202, Venice, CA, 90291, US

(73) Assignee: INDUSTRIAL TECHNOLOGY RESEARCH INSTITUTE, Hsinchu (TW)

(21) Appl. No.: 12/410,373

(22) Filed: Mar. 24, 2009

(30) Foreign Application Priority Data
Dec. 12, 2008 (TW) TW097148471

(51) Int. Cl. A61M 27/00

(52) U.S. Cl. 604/541

(57) ABSTRACT

A collecting device is provided and utilized to collect a fluid, such as waste liquid or blood from a wound. The collecting device includes a first guiding element, an absorbing element and a second guiding element. The absorbing element includes a plurality of absorbing units disposed on the first guiding element. When the fluid is initially absorbed by at least one of the plurality of absorbing units, the fluid is then sequentially absorbed by the rest of the absorbing units along a predetermined path. The second guiding element is disposed on the first guiding element. The fluid absorbed by the plurality of absorbing units of the absorbing element is impeded to travel along the predetermined path by impediments of the second guiding element.

### CROSS REFERENCE TO RELATED APPLICATIONS

This Application claims priority of Taiwan Patent Application No. 97148471, filed on Dec. 12, 2008, the entirety of which is incorporated by reference herein.

### BACKGROUND OF THE INVENTION

1. Field of the Invention

The present invention relates to a fluid processing system, and in particular relates to a fluid processing system and a collecting device thereof capable of absorbing waste liquid or blood originating from a wound.

2. Description of the Related Art

For negative pressure wound therapy (NPWT), a simple vacuum pump, an occlusive transparent film, a drainage tube and biologically compatible porous material are utilized to form a negative environment to absorb waste fluid (e.g., pus) and contain substances from a wound. NPWT therapeutically helps to keep the wound under a moist environment, so that blood can circulate to speed up recovery of the wound.

For example, in U.S. Pat. No. 5,549,584 rendered by Kendall Company in 1996, an apparatus for removing fluid from a wound is disclosed. A manual pump is utilized to store the waste fluid in a collecting bag. In U.S. Pat. No. 5,636,643 rendered by Wake Forest University in 1995, a wound treatment employing reduced pressure is disclosed, wherein waste fluid is stored in a container. In U.S. Publication No. 20060015087 rendered by James Robert Risk JR, et al, in 2006, a waste container for negative pressure therapy is disclosed, wherein waste fluid is also stored in a container.

In U.S. Pat. No. 6,648,862 rendered by Spheric Products Company in 2003, a portable vacuum desiccator is disclosed, wherein waste fluid is absorbed by an absorber received in the chamber of a cassette.

FIG. 1A is a schematic view of a conventional negative pressure wound therapy (NPWT) device E, and FIG. 1B is a schematic block diagram of the conventional NPWT device E of FIG. 1A. A wound C′ is connected to a canister e1 by a conduit p1, and the canister e1 is connected to a vacuum pump e2 by another conduit p2. The vacuum pump e2 is electrically connected to a battery e3. However, because the length of the negative pipeline of the NPWT device E is too long, the total size thereof cannot be effectively reduced, thus, decreasing portability, and the time for loading and required power of the vacuum pump e2 are relatively high.

### BRIEF SUMMARY OF THE INVENTION

In view of the problems of the conventional arts, the invention provides a fluid processing system to process and collect a fluid (e.g., waste liquid or blood) originating from a source (e.g., wound). The fluid processing system comprises an absorbing device, a driving device and a collecting device. A negative environment formed by the absorbing device and the driving device is utilized to transmit the fluid. The absorbing device disposed on the source is utilized to absorb the fluid. The driving device connected to the absorbing device is utilized to move the fluid collected by the absorbing device. The collecting device is utilized to absorb the fluid moved by the driving device.

The collecting device comprises a first guiding element, an absorbing element and a second guiding element. The first guiding element is connected to the driving device. The absorbing element comprises a plurality of absorbing units disposed on the first guiding element. The fluid travels along a predetermined path from at least one of the plurality of absorbing units to sequentially pass through the rest of the plurality of absorbing units. The second guiding element is disposed on the first guiding element. The fluid passing through the plurality of absorbing units of the absorbing element is impeded by the second guiding element to travel along the predetermined path.

The driving device is disposed between the absorbing device and the collecting device. The driving device is a vacuum pump.

In one embodiment, the first guiding element of the collecting device is a water-guiding layer (e.g., an anticontaminate or cotton paper). The plurality of absorbing units of the absorbing element of the collecting device is formed by polymer material. The second guiding element of the collecting device is a water-separating layer made by a plastic material.

In one embodiment, the collecting device further comprises a base portion to enclose the first guiding element and the absorbing element therein. The base portion comprises a first layer and a second layer, the first guiding element and the absorbing element are enclosed between the first and second layers of the base portion, and the first and second layers of the base portion are connected with each other to form the second guiding element. The first and second layers of the base portion are connected with each other by a thermal bonding method, and the part of the first and second layers of the base portion connected with each other forms the second guiding element and the predetermined path. The thermal bonding method is an ultrasonic or radio-frequency bonding method. The first and second layers of the base portion are made of plastic materials.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1A is a schematic view of a conventional negative pressure wound therapy (NPWT) device;

FIG. 1B is a schematic block diagram of the conventional negative pressure wound therapy (NPWT) device of FIG. 1A;

FIG. 2A is a schematic view of a fluid processing system of an embodiment of the invention;

FIG. 2B is a schematic block diagram of the fluid processing system of FIG. 2A;

FIG. 3 is a schematic view of a collecting device of an embodiment of the invention;

FIG. 4 is a partially enlarged view of the collecting device of FIG. 3;

FIG. 5 is a sectional view of line (a1-a1) in FIG. 4; and

FIG. 6 is a schematic view of a collecting device of another embodiment of the invention.

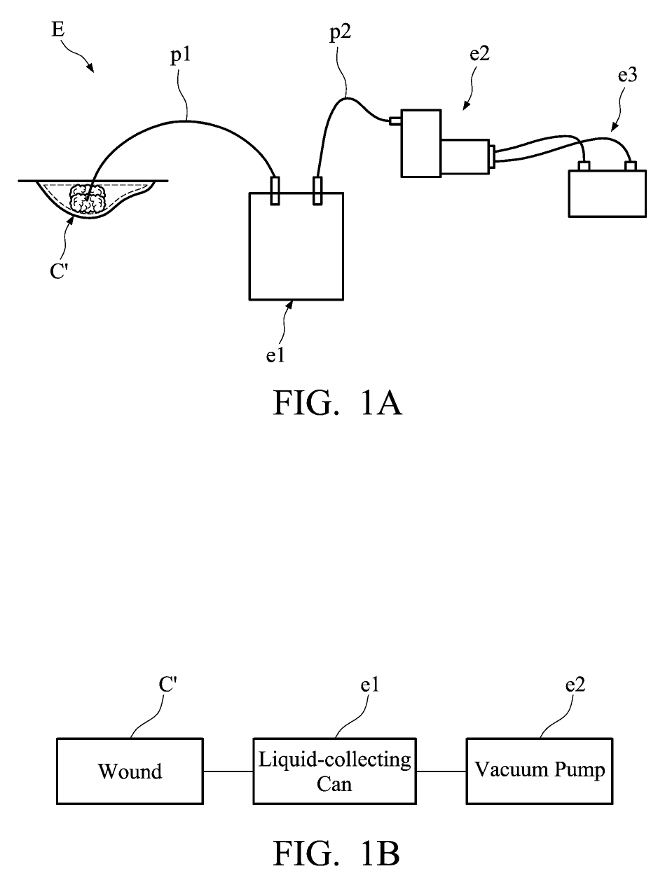

### DETAILED DESCRIPTION OF THE INVENTION

The following description is of the be-contemplated mode of carrying out the invention. This description is made for the purpose of illustrating the general principles of the invention and should not be taken in a limiting sense. The scope of the invention is best determined by reference to the appended claims.

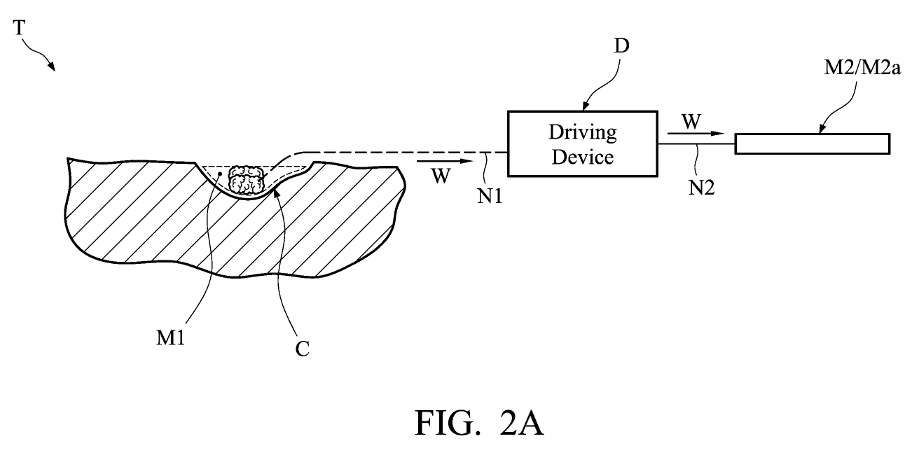

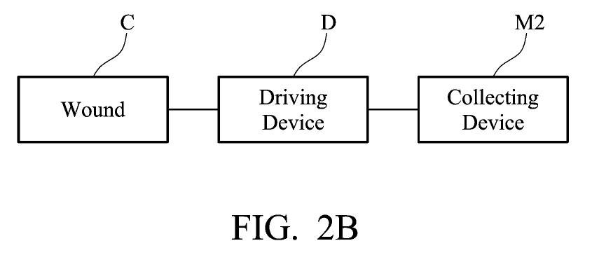

FIG. 2A is a schematic view of a fluid processing system T of an embodiment, and FIG. 2B is a schematic block diagram of the fluid processing system T of FIG. 2A. The fluid processing system T is utilized to process a fluid W (e.g., waste liquid) originating from a source C (e.g., wound). In the following description, the source C is the wound C and the fluid W is the waste liquid W.

The fluid processing system T comprises an absorbing device M1, a driving device D and a collecting device M2.

The absorbing device M1 disposed at the wound C is utilized to collect the waste liquid W. In this embodiment, the absorbing device M1 is assembled by an occlusive transparent film, a flexible conducting device and a biologically compatible porous material (not shown).

The driving device D is detachably disposed between the absorbing device M1 and the collecting device M2. With the driving device D, the waste liquid W collected by the absorbing device M1 is transmitted to the driving device D via a first pipeline N1, and then the waste liquid W is transmitted to the collecting device M2 via a second pipeline N2. The collecting device M2 is utilized to collect the waste liquid W moved by the driving device D. That is, with the absorbing device M1 and the driving device D, a negative environment is formed inside the wound C. In this embodiment, the driving device D is a vacuum pump.

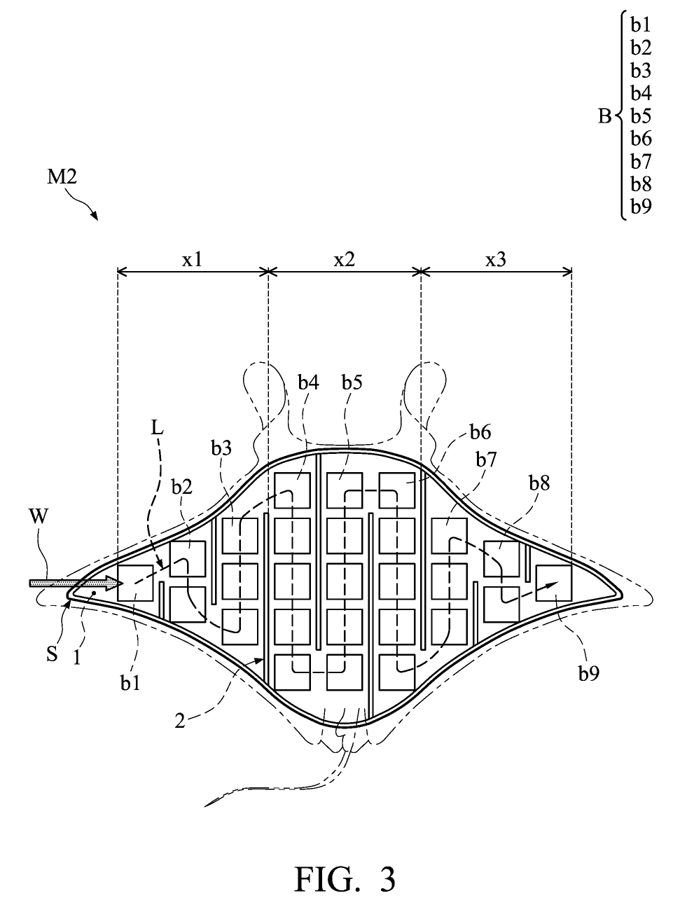

FIG. 3 is a schematic view of the collecting device of an embodiment. The collecting device M2 comprises a base portion S, a first guiding element 1, an absorbing element B and a second guiding element 2. The first guiding element 1 and the absorbing element B are enclosed by the absorbing element B. In this embodiment, the collecting device M2 is divided into three regions x1, x2 and x3.

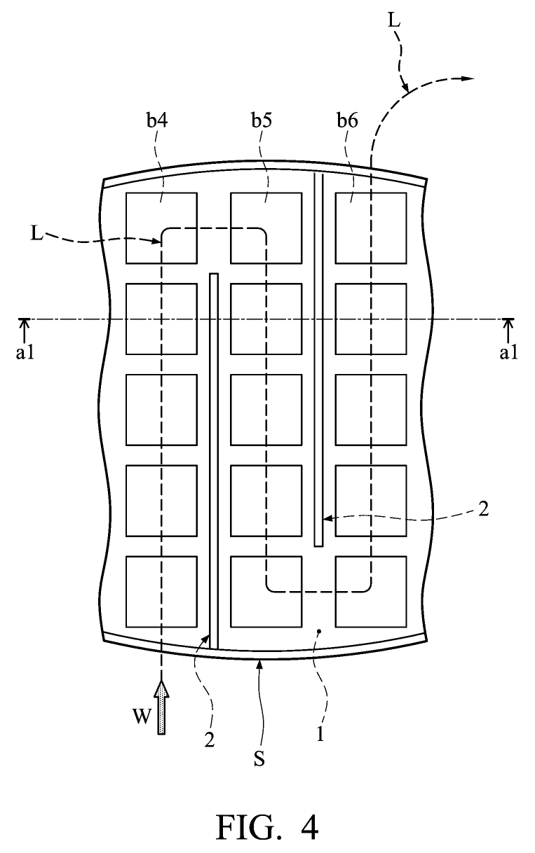

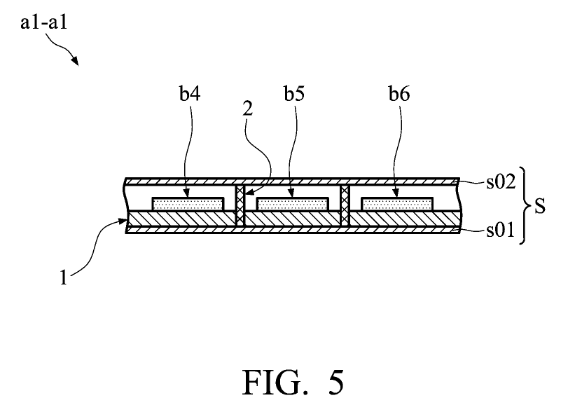

FIG. 4 is an enlarged view of the region x2 of the collecting device M2 of FIG. 3, and FIG. 5 is a sectional view of line a1-a1 in FIG. 4. In FIGS. 4 and 5, the base portion S comprises a first layer s01 and a second layer s02. The first guiding element 1 and the absorbing element B are enclosed between the first and second layers s01 and s02 of the base portion S. In this embodiment, the first and second layers s01 and s02 of the base portion S are two transparent plastic sheets or materials, and the outline of the absorbing element B is formed as a polygon (similar to a skate-like shape) composed by one similar rectangular region x2 and two triangular regions x1 and x3, wherein the similar rectangular region x2 is located between the triangular regions x1 and x3. Although the outline of the absorbing element B is formed as the skate-like shape in this embodiment, it is not to be limited thereto. To the contrary, the outline of the absorbing element B may be altered according to the requirements.

The first guiding element 1 is a water-guiding layer connected to the driving device D and substantially provided with a predetermined path L. In this embodiment, the first guiding element 1 can be an anticontaminate or cotton paper.

In FIG. 3, the absorbing element B comprises a plurality of absorbing units (or small unit liquid-collecting packs) b1-b9 disposed on the first guiding element 1. The waste liquid W travels along the predetermined path L from the absorbing unit b1 to sequentially pass through the absorbing units b2-b9. In this embodiment, the plurality of absorbing units b1-b9 of the absorbing element B are made of polymer, and are substantially formed as rectangular shapes and arranged in a matrix.

The second guiding element 2 is disposed on the first guiding element 1 and next to the plurality of absorbing units b1-b9 of the absorbing element B, thereby impeding the waste liquid W to pass through the absorbing units b1-b9 of the absorbing element B by traveling along the predetermined path L. Thus, the waste liquid W sequentially passes through the absorbing units b1-b9. When the first and second layers s01 and s02 of the base portion S are connected by a thermal bonding method (e.g., ultrasonic or radio-frequency bonding method), the bonded part of the first and second layers s01 and s02 of the base portion S connected with each other form the second guiding element 2 and the predetermined path L. That is, the first guiding element 1 is clamped between the bonded part of the first and second layers s01 and s02 of the base portion S, so that the second guiding element 2 serves as a water-separating layer to guide the waste liquid W to travel along the predetermined path L. In this embodiment, it is understood that the second guiding element 2 and the first and second layers s01 and s02 of the base portion S are made of the same material (plastic material) because the second guiding element 2 is thermally formed by the bonded part of the first and second layers s01 and s02 of the base portion S, and the second guiding element 2 comprises a plurality of bonded parts. In other embodiments, the second guiding element can be a plurality of protrusions directly formed on the first guiding element.

With the first guiding element 1 (water-guiding layer) and the second guiding element 2 (water-separating layer), when the waste liquid W, originating from the absorbing device M1 and driven by the driving device D, enters the collecting device M2, the waste liquid W sequentially passes through the absorbing units b1-b9 of the three regions x1, x2 and x3 by traveling along the predetermined path L.

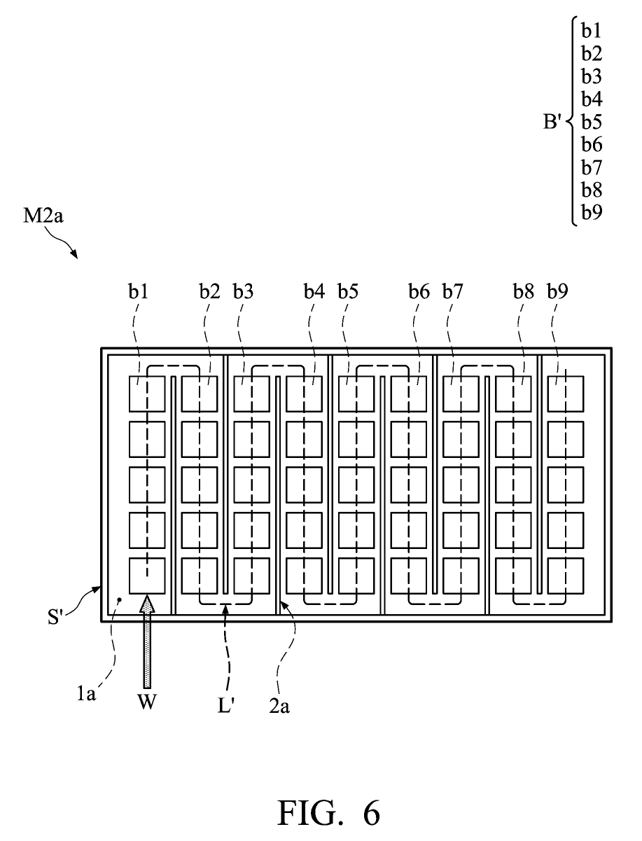

FIG. 6 is a schematic view of a collecting device M2a of another embodiment. The collecting device M2a differs from the collecting device M2 in FIG. 3 in that the base portion S′ and the first guiding element 1a are formed in rectangular shapes, and the plurality of absorbing units b1-b9 of the absorbing element B′ are arranged in a matrix. Thus, impeded by the second guiding element 2a, the waste liquid W sequentially passes through the absorbing units b1-b9 by traveling along the predetermined path L′. Basically, the collecting device M2 of FIG. 3 and the collecting device M2a of FIG. 6 are provided with a particular area to absorb the waste liquid and are capable of being adapted to the shape of a human body. Thus, the profile of the collecting device is not limited to the disclosed embodiments, and the profile of the collecting device can be arbitrarily altered according to the requirements.

With the fluid processing system and the collecting devices of the described embodiments, the length of the negative pipes can be shortened, and the time for loading and power of the negative pump can be decreased. Additionally, the amount of the collected waste liquid can be effectively absorbed by the liquid-collecting packs (absorbing units) and precisely estimated. Further, once the waste liquid enters the liquid-collecting packs (absorbing units) of the absorbing element, the waste liquid absorbed by the liquid-collecting packs does not ooze or overflow to contaminate the environment even if the absorbing element is squeezed or compressed. With the transparent first and second layers of the base portion, the condition of the collecting device can be inspected by users and thus effectively prompt replacement of the saturated liquid-collecting packs. Additionally, due to the flexibility and thinness of the collecting device, the portable collecting device can be worn comfortable by users.

While the invention has been described by way of example and in terms of several embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A collecting device utilized to collect a fluid, comprising:
a first guiding element;
an absorbing element comprising a plurality of absorbing units disposed on the first guiding element, the fluid traveling along a predetermined path from at least one of the plurality of absorbing units to sequentially pass through the rest of the plurality of absorbing units; and
a second guiding element disposed on the first guiding element, the fluid passing through the plurality of absorbing units of the absorbing element being impeded by the second guiding element to travel along the predetermined path.

2. The collecting device as claimed in claim 1, wherein the first guiding element comprises a water-guiding layer.

3. The collecting device as claimed in claim 1, wherein the plurality of absorbing units of the absorbing element comprise polymer material.

4. The collecting device as claimed in claim 1, wherein the second guiding element comprises a water-separating layer.

5. The collecting device as claimed in claim 4, wherein the water-separating layer comprises a plastic material.

6. The collecting device as claimed in claim 1 further comprising a base portion to enclose the first guiding element and the absorbing element therein.

7. The collecting device as claimed in claim 6, wherein the base portion comprises a first layer and a second layer, the first guiding element and the absorbing element are enclosed between the first and second layers of the base portion, and the first and second layers of the base portion are connected with each other to form the second guiding element.

8. The collecting device as claimed in claim 7, wherein the first and second layers of the base portion are connected with each other by a thermal bonding method, and the part of the first and second layers of the base portion connected with each other forms the second guiding element and the predetermined path.

9. The collecting device as claimed in claim 8, wherein the thermal bonding method comprises an ultrasonic or radio-frequency bonding method.

10. The collecting device as claimed in claim 7, wherein the first and second layers of the base portion comprise plastic materials.

11. The collecting device as claimed in claim 5, wherein the base portion comprises a plastic material.

12. A fluid processing system utilized to process a fluid originating from a source, comprising:
an absorbing device disposed on the source to absorb the fluid;
a driving device connected to the absorbing device to move the fluid collected by the absorbing device; and
a collecting device utilized to absorb the fluid moved by the driving device, comprising:
a first guiding element connected to the driving device;
an absorbing element comprising a plurality of absorbing units disposed on the first guiding element, the fluid traveling along a predetermined path from at least one of the plurality of absorbing units to sequentially pass through the rest of the plurality of absorbing units; and
a second guiding element disposed on the first guiding element, the fluid passing through the plurality of absorbing units of the absorbing element being impeded by the second guiding element to travel along the predetermined path.

13. The fluid processing system as claimed in claim 12, wherein the driving device is disposed between the absorbing device and the collecting device.

14. The fluid processing system as claimed in claim 13, wherein the driving device comprises a vacuum pump.

15. The fluid processing system as claimed in claim 12, wherein the first guiding element of the collecting device comprises a water-guiding layer.

16. The fluid processing system as claimed in claim 12, wherein the plurality of absorbing units of the absorbing element of the collecting device comprise polymer material.

17. The fluid processing system as claimed in claim 12, wherein the second guiding element of the collecting device comprises a water-separating layer.

18. The fluid processing system as claimed in claim 17, wherein the water-separating layer comprises a plastic material.

19. The fluid processing system as claimed in claim 12, wherein the collecting device further comprises a base portion to enclose the first guiding element and the absorbing element therein.

20. The fluid processing system as claimed in claim 19, wherein the base portion comprises a first layer and a second layer, the first guiding element and the absorbing element are enclosed between the first and second layers of the base portion, and the first and second layers of the base portion are connected with each other to form the second guiding element.

21. The fluid processing system as claimed in claim 20, wherein the first and second layers of the base portion are connected with each other by a thermal bonding method, and parts of the first and second layers of the base portion that are connected with each other form the second guiding element and the predetermined path.

22. The fluid processing system as claimed in claim 21, wherein the thermal bonding method comprises an ultrasonic or radio-frequency bonding method.

23. The fluid processing system as claimed in claim 20, wherein the first and second layers of the base portion comprise plastic materials.

24. The fluid processing system as claimed in claim 19, wherein the base portion comprises a plastic material.

25. The fluid processing system as claimed in claim 12, wherein the absorbing device and the driving device form a negative environment to conduct the fluid.

26. The fluid processing system as claimed in claim 12, wherein the source comprises a wound, and the fluid comprises waste liquid or blood generated from the wound.
